(19) United States
(12) Patent Application Publication
(10) Pub. No.: US 2006/0159663 A1
(43) Pub. Date: Jul. 20, 2006

(54) Growth factor encapsulation system for enhancing bone formation

(76) Inventors: Helen H. Lu, New York, NY (US); Regina Landesberg, Stamford, CT (US); Jennifer M. Vo, Hawthorne, NJ (US); Rick Tsay, Los Angeles, CA (US); Hsin-I Peng, Shanghai (CN)

Correspondence Address: COOPER & DUNHAM, LLP, 1185 AVENUE OF THE AMERICAS, NEW YORK, NY, 10036, US

(21) Appl. No.: 11/194,030

(22) Filed: Jul. 29, 2005

Related U.S. Application Data
(60) Provisional application No. 60/592,442, filed on Jul. 30, 2004.

(51) Int. Cl. A61K 38/18; A61K 35/14; A61K 9/48

(52) U.S. Cl. 424/93.7; 424/451; 514/12

(57) ABSTRACT

This invention provides articles of manufacture, compositions of matter and methods related to a growth factor encapsulation system for enhancing bone formation.

This application claims the benefit of U.S. Provisional Application No. 60/592,442, filed Jul. 30, 2004, the contents of which are incorporated herein by reference into the subject application.

Throughout this application, various publications are referred to by Arabic numerals within parentheses. Full citations for these publications are presented immediately before the claims. Disclosures of these publications in their entireties are hereby incorporated by reference into this application in order to more fully describe the state of the art to which this invention pertains.

### BACKGROUND OF THE INVENTION

PRP is known to contain a number of autologous thrombocyte growth factors that may aid in the acceleration of bone regeneration (1). These growth factors include platelet-derived growth factor (PDGF), transforming growth factors β1 and β2 (TGF-β1 and TGF-β2), insulin-like growth factor (IGF), epidermal growth factor (EGF), epithelial cell growth factor (ECGF), and a hepatocyte growth factor (HGF) (1). Although there are many growth factors/cytokines that play a role during bone graft healing, PDGF and TGF-β1 are known to be produced by platelets and released during degranulation. PDGF stimulates mitogenesis of osteoblastic precursors while TGF-β1 stimulates proliferation and collagen synthesis by osteoblasts and osteoblast precursors (2, 3). PRP gel has numerous applications, such as cardiac and neurosurgical areas, and most recently, it has been used as an adhesive with cancellous bone particles in oral and maxillofacial surgery bone grafting procedures (2). However, basic data and exhaustive studies on thrombocyte growth factor levels in PRP have not been determined.

### SUMMARY OF THE INVENTION

This invention provides an article of manufacture comprising a capsule of protein-permeable material having platelet-rich plasma therein.

This invention further provides an article of manufacture comprising a porous bead having releasably contained therein (i) platelet-rich plasma and/or (ii) a growth factor.

This invention further provides a composition of matter comprising (a) a capsule of protein-permeable material having a growth factor therein, (b) a porous bead having a growth factor releasably contained therein, and (c) a gel comprising platelet-rich plasma and a bone regeneration-facilitating material.

This invention further provides a method for making an article of manufacture comprising a capsule of protein-permeable material having platelet-rich plasma therein, which method comprises admixing platelet-rich plasma dropwise, under suitable conditions, with a material which, when solidified under such conditions, forms a protein-permeable capsule.

This invention further provides a method for facilitating bone formation in a subject comprising delivering to a bone formation-requiring site in the subject an article of manufacture of comprising a porous bead having autologous platelet-rich plasma releasably contained therein.

This invention further provides a method for delivering a platelet-originating growth factor to a subject at a location in the subject where delivery of the growth factor is desired comprising delivering to the site in the subject a capsule of protein-permeable material having autologous platelet-rich plasma therein, so as to permit the platelet-originating growth factor to be released from the platelets in the platelet-rich plasma and then be released from the capsule, thereby delivering the platelet-originating growth factor to the subject at the location where delivery of the growth factor is desired.

This invention further provides a method for delivering a platelet-originating growth factor to a subject at a location in the subject where delivery of the growth factor is desired comprising delivering to the site in the subject a porous bead having autologous platelet-rich plasma releasably contained therein, so as to permit the platelet-originating growth factor to be released from the platelets in the platelet-rich plasma and then be released from the bead, thereby delivering the platelet-originating growth factor to the subject at the location where delivery of the growth factor is desired.

Finally, this invention provides an article of manufacture comprising a packaging material having therein, in separate compartments, calcium and a material which, when solidified under suitable conditions, forms a protein-permeable capsule.

### BRIEF DESCRIPTION OF THE FIGURES

FIG. 1 (a) PRP/4% CaCl₂/10% dextran encapsulated within 0.5% alginate. PRP and CaCl₂ were combined in a 1:1 ratio. Dropping height of PRP was maintained at approximately 4 inches above the alginate solution. (b) PRP/4% CaCl₂/10% dextran encapsulated within 1% alginate (20×). PRP and CaCl₂ were combined in a 1:3 ratio. Dropping height of PRP was maintained at approximately 12 inches above the alginate solution. Note that the capsule walls are in tact and representative of a spherical morphology. PRP appears to be uniformly dispersed throughout the capsule.

FIG. 2 Schematic of apparatus used to encapsulate PRP. With a syringe clamped to a stand, PRP/CaCl₂ is dropped into 1% alginate via a 26Gb½ syringe needle.

FIG. 3 Schematic of capsules in 24-well plate at each time point.

FIG. 4 Seven capsules were incubated in unsupplemented DMEM for 1, 3, and 7 days (n=3).

FIG. 5 Capsules were uniform in size (4.22±0.37 mm×3.09±0.21 mm) with uniform wall thickness of 0.61±0.05 mm. Morphology and membrane integrity were maintained over time.

FIG. 6 Temporal effects of encapsulation on PDGF-AB release. *Statistical significance between supernatant with PRP at day 7 and all other time points (p<0.05).

FIG. 7 Effects of substrate on PDGF-AB released per μl of PRP at 24 hours. *Statistical significance between thrombin group and all other groups (p<0.05); **Statistical significance between TRAP group and all other groups (p<0.05).

FIG. 8 Logarithmic release kinetics of PDGF-AB from alginate capsules over 7 days.

FIG. 9 Bone regeneration cascade.

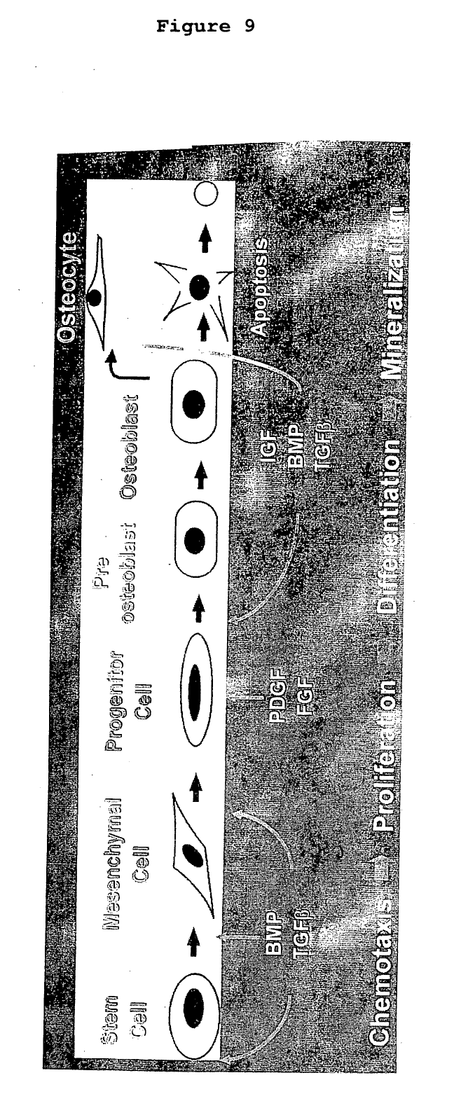

FIG. 10 Hydrogel-alginate can be extracted from the cell walls of brown seaweed and comprises linear co-polymers of 1,4-linked D-mannuronic acid (M), L-guluronic (G). Structures may vary depending on the sequence of the monomer (MM, GG, MG) and may range in size from 50 to 500 kDa. Hydrogel alginate is used in food and pharmaceutical industries as a thickener.

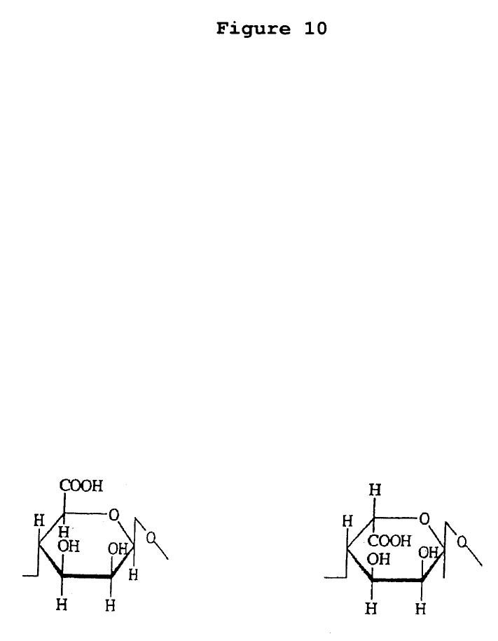

FIG. 11 Crosslinking of Alginate. Gelation is mediated by divalent cations (Mg²⁺, Ca²⁺). In external gelation, the alginate is dropped into an aqueous calcium chloride solution. Internal gelation is performed by physically dispersing solid calcium salt particles in alginate solution.

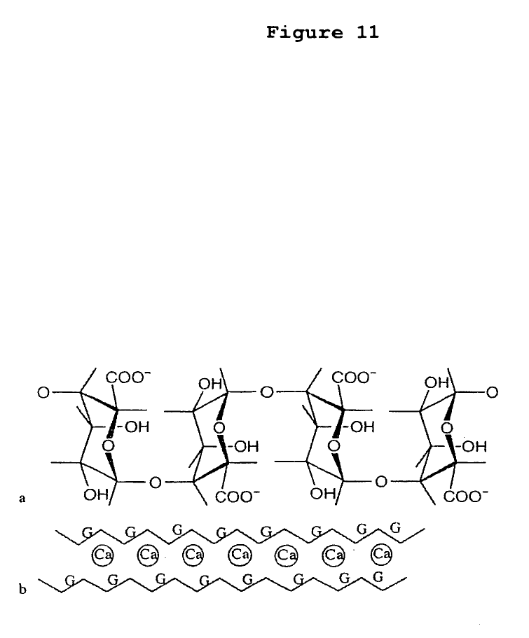

FIG. 12 Fabrication of Alginate Beads. PRP is re-suspended in 2% alginate solution and dispensed drop-wise via a 26½-gauge needle into a 6% CaCl₂ solution. The control beads without PRP were also fabricated in the same manner.

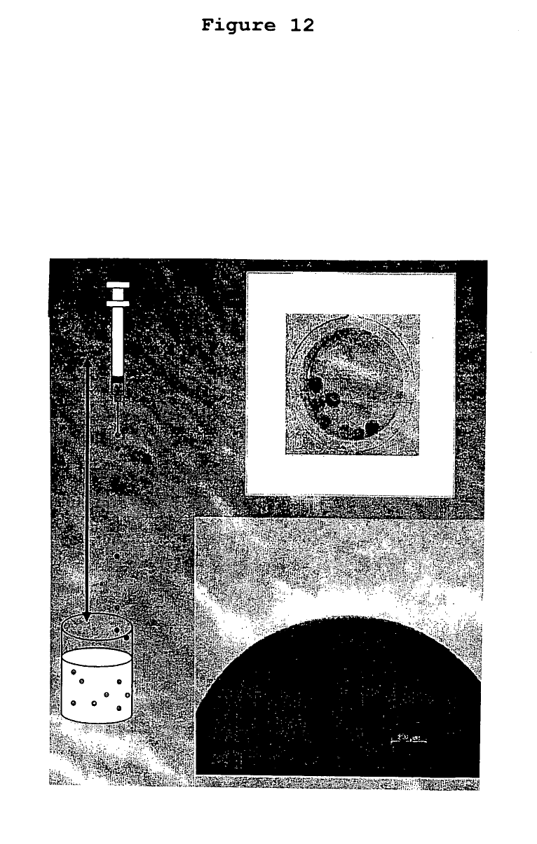

FIG. 13 Fabrication of Alginate Capsules. PRP is re-suspended in 6% CaCl₂ solution and dispensed drop-wise via a 26½-gauge needle into stirring 1% alginate solution. The Capsules are then removed, transferred to 6% CaCl₂, and washed with DMEM. The control capsules without PRP were also fabricated in the same manner.

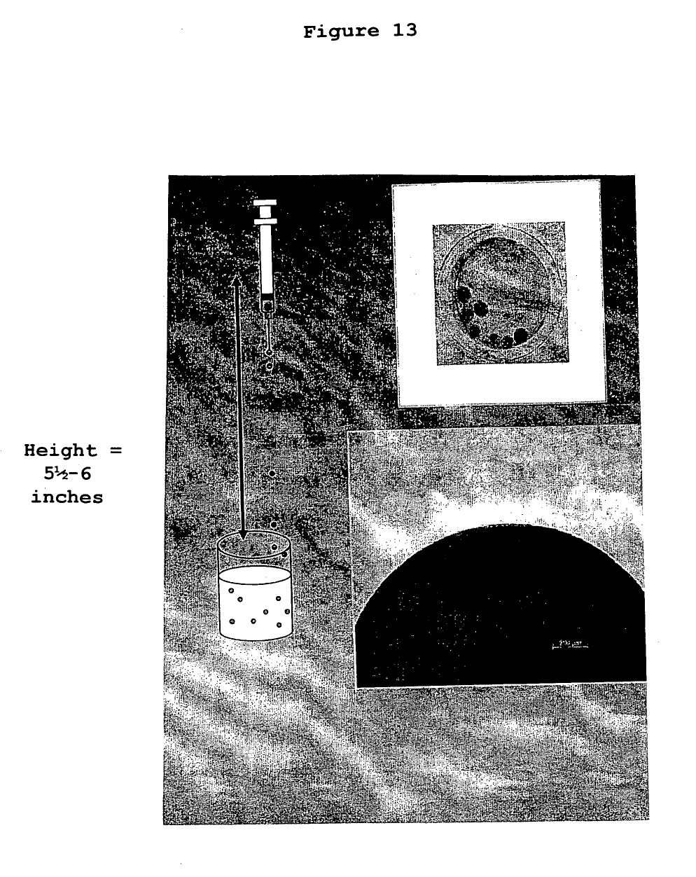

FIG. 14 Optimization of Capsules. Factors affecting the optimization of capsules include stirring speed, dropping height and the concentration of cationic and anionic solutions.

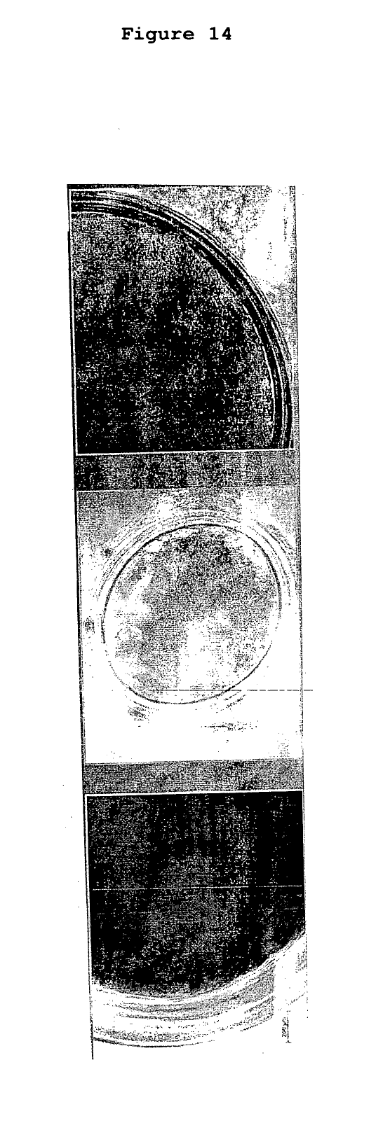

FIG. 15 PDGF Release from Alginate Beads. PDGF-AB within the bead was detectable only in the Day 0 sample. PDGF-AB in the supernatant was minimally detectable at all time points. Dilution of the beads had a minimal effect. PDGF may bind to the alginate matrix in the presence of cations.

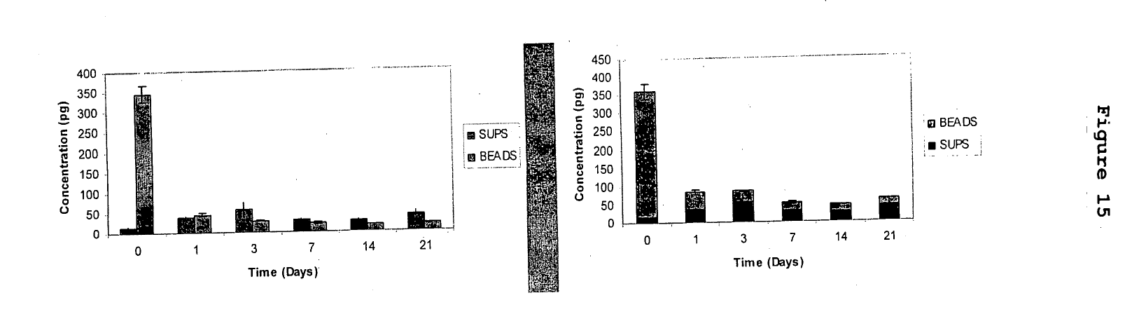

FIG. 16 Effects of Substrate on PDGF Release. There is a significant reduction in PDGF release at 24 hours when encapsulated in alginate. The mode of PDGF retention modulates PDGF release. The alginate beads retained more PDGF compared to the capsules.

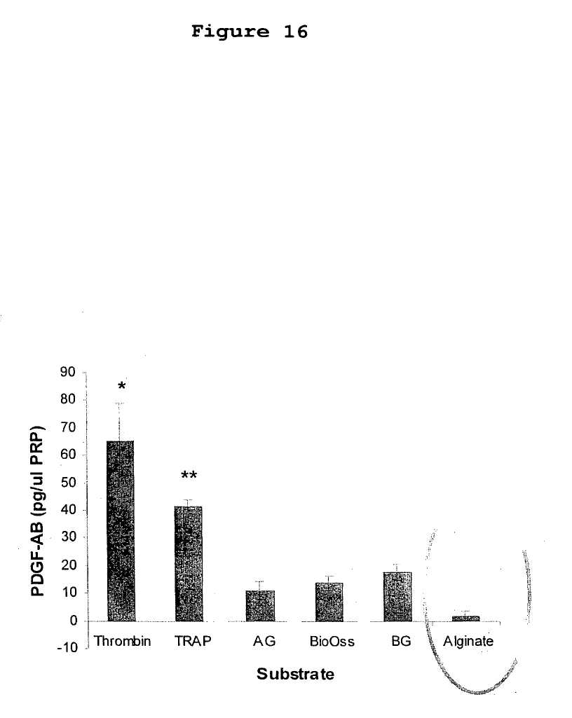

FIG. 17 PDGF—Beads vs. Capsule. Experiments show a greater release of PDGF from capsules over time, peaking at Day 14.

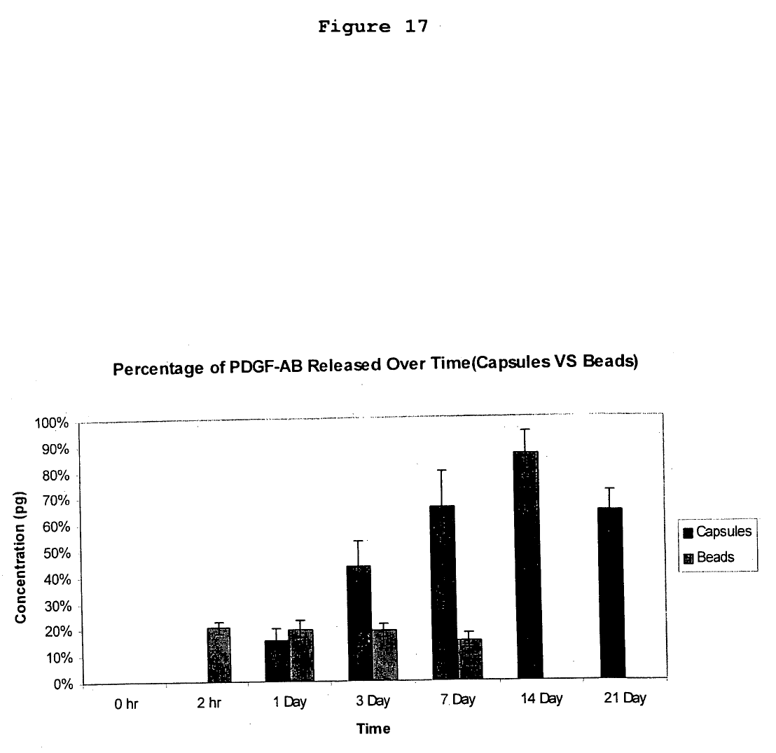

FIG. 18 TGF-β Release from Alginate Beads. TGF-β levels within beads decreased as it was released into the media. TGF-β levels in supernatant increased as incubation time increased.

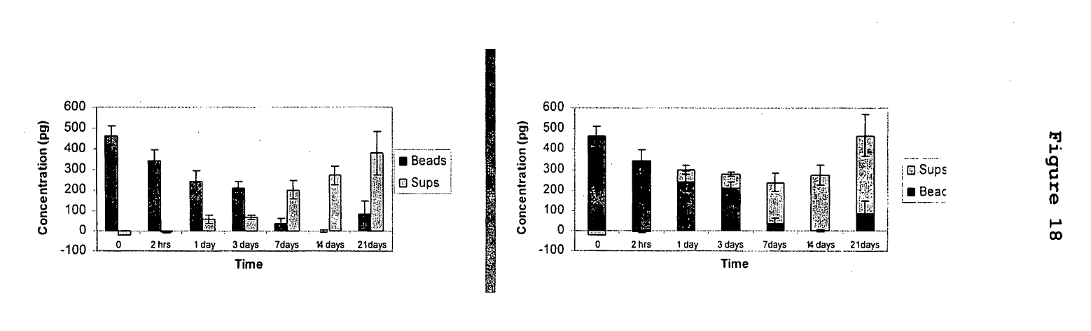

FIG. 19 TGF-β Release from Capsules. TGF-β was not released from capsules until Day 7.

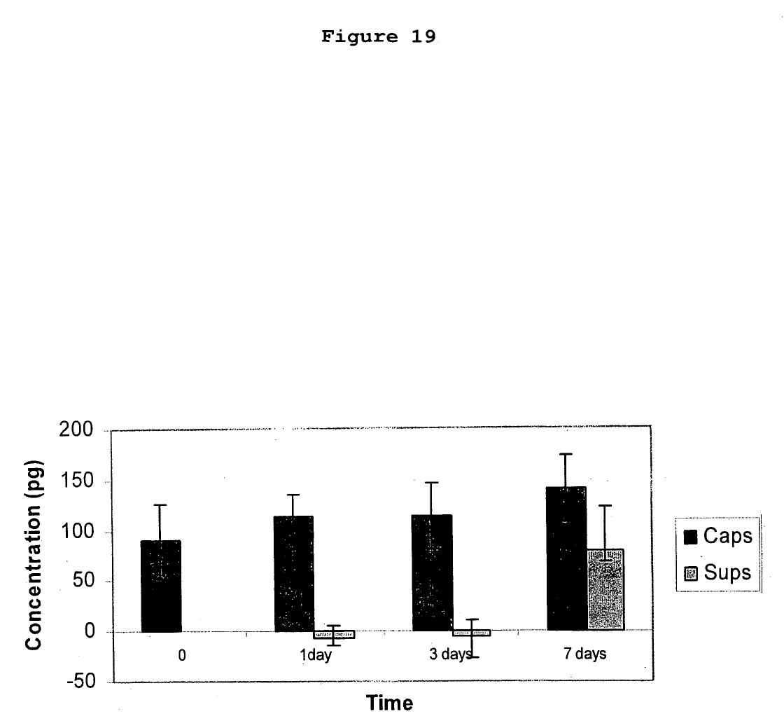

FIG. 20 TGF-β—Beads vs. Capsules. Experiments show a greater release of TGF-β from beads compared to capsules.

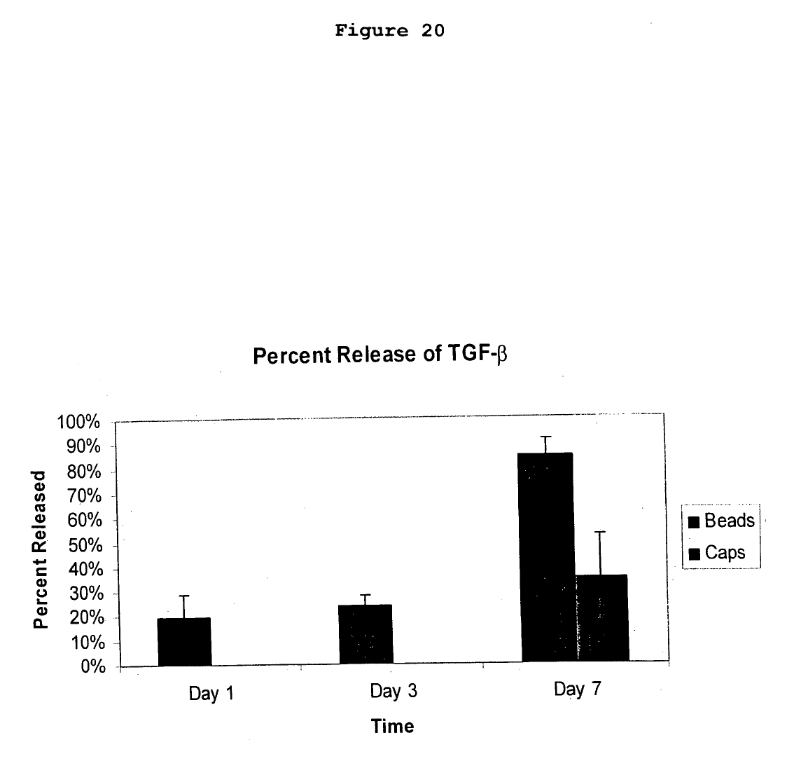

FIG. 21 TGF-β—Beads vs. Capsules. Experiments show a greater release of TGF-β from beads compared to capsules. TGF-β release from beads peaked on Day 7.

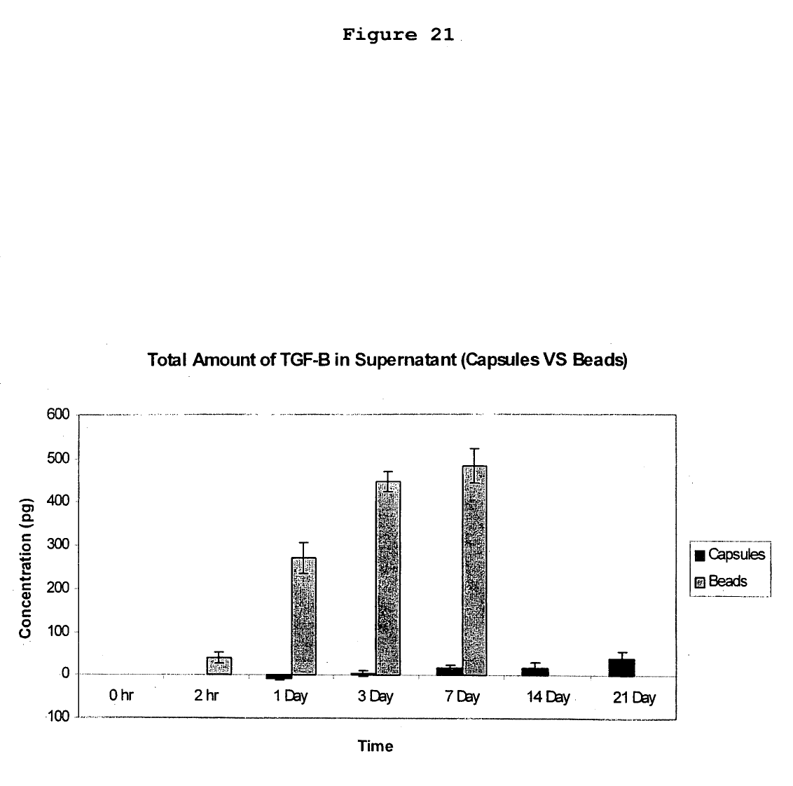

FIG. 22 IGF Release from Beads. Experiments show a gradual release of IGF from beads, which plateaued after 24 hours.

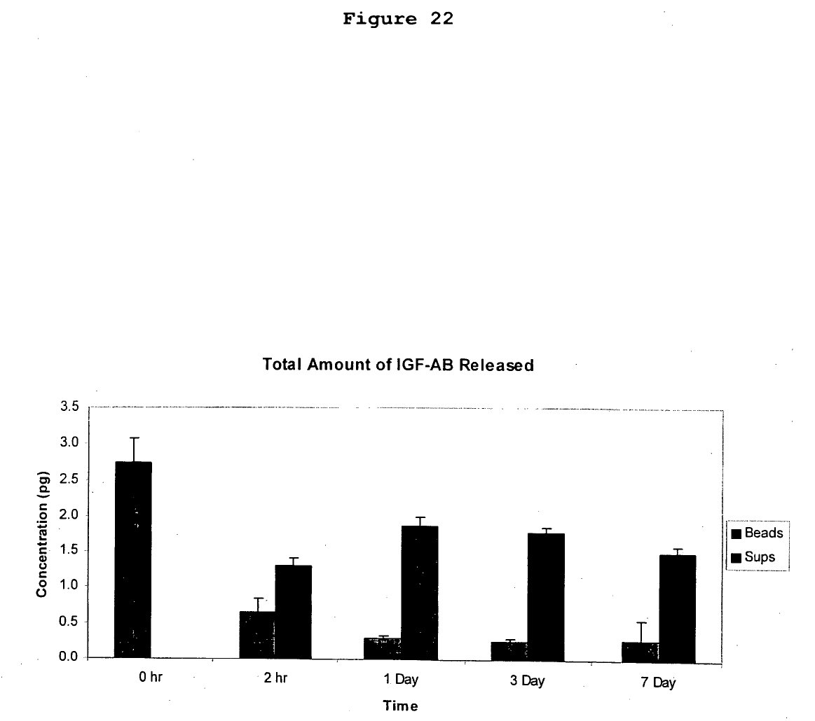

FIG. 23 IGF Release from Capsules. Experiments show a constant release of IGF from capsules.

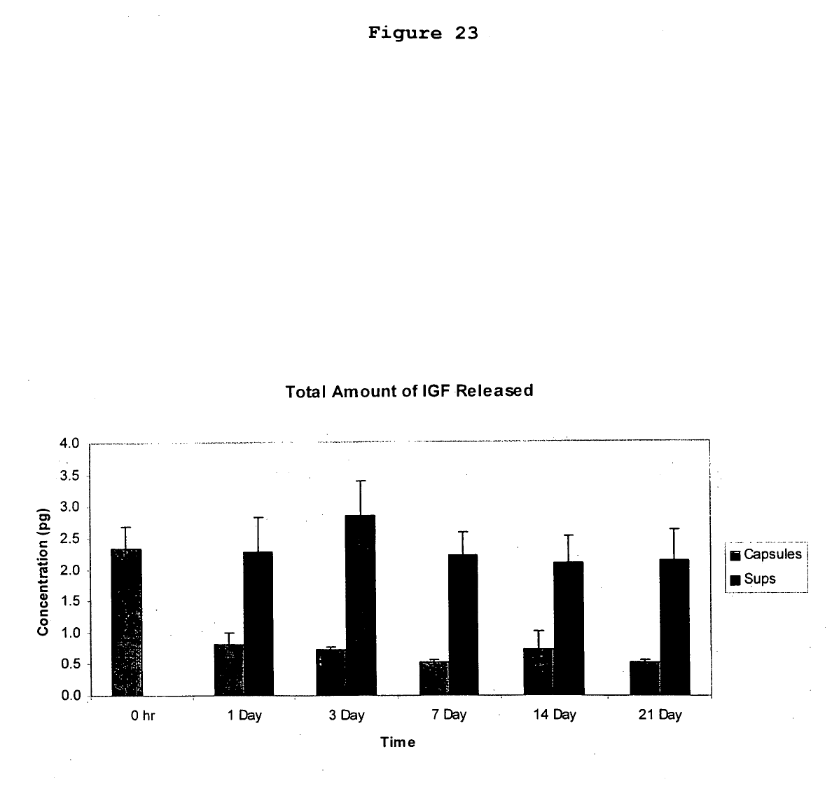

FIG. 24 IGF—Bead vs. Capsule. Experiments show greater IGF release from capsules than IGF release from beads. Results also indicate a similar trend in constant release of IGF.

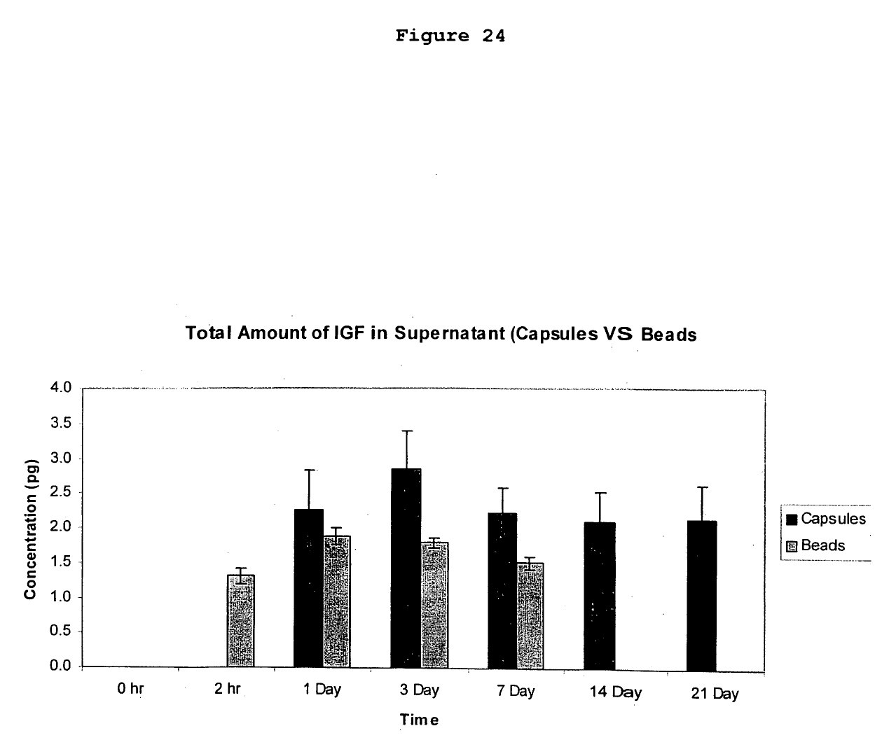

FIG. 25 Controlled, Staged Release of PRP-Derived Growth Factors. The controlled release of PRP-derived growth factors can be achieved by PRP encapsulation (capsules) and embedding (beads) within a hydrogel. A novel hydrogel delivery system permits prolonged and modulated, staged release of growth factors relevant for bone regeneration.

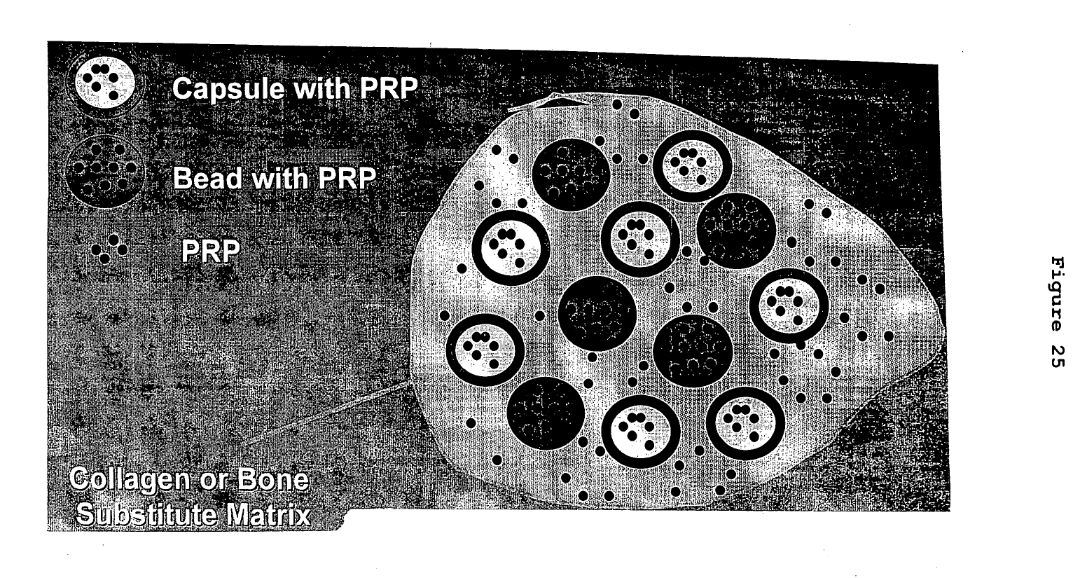

FIG. 26 Monomer structure of chitosan.

FIG. 27 Cell Number. Alginate+PRP beads have greater effects on proliferation of human osteoblast-like cells.

FIG. 28 ALP Activity (Quantitative). Maximum ALP activity is observed at Day 21.

FIG. 29 Mineralization (Quantitative). Mineralization increases over time.

FIG. 30 Formation of Chitosan Beads.

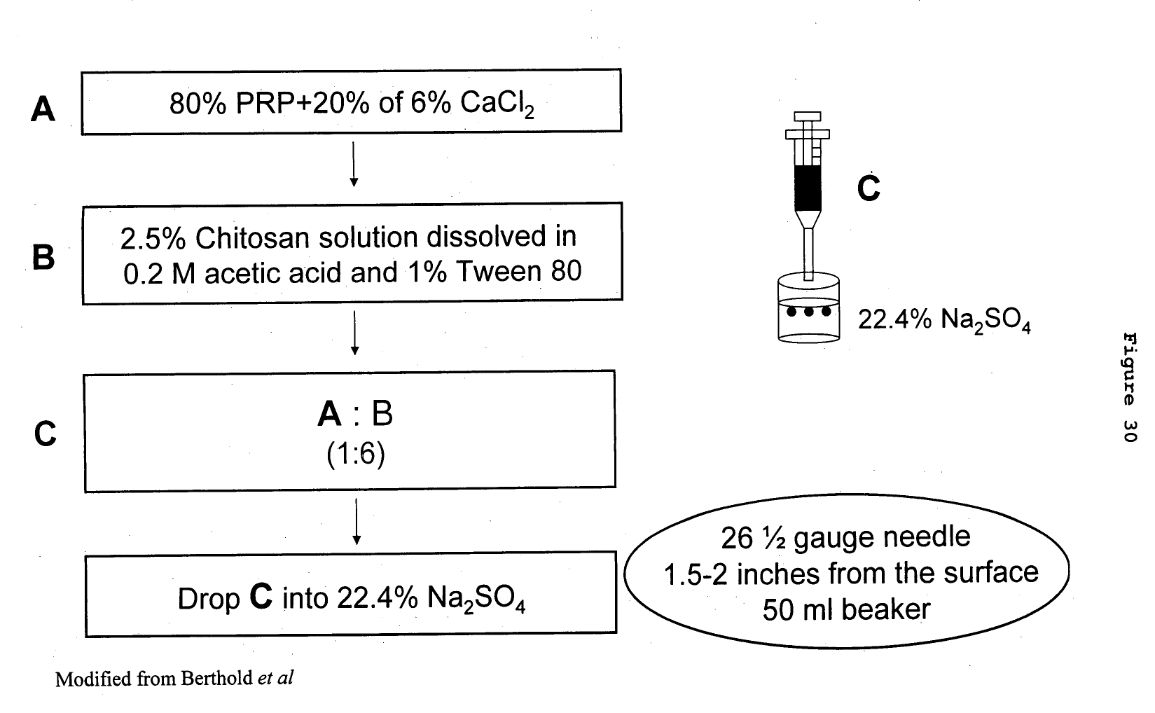

FIG. 31 Chitosan Beads. 1:3 ration of PRP to 2.5% Chitosan solution (Φ 2.5-3 mm).

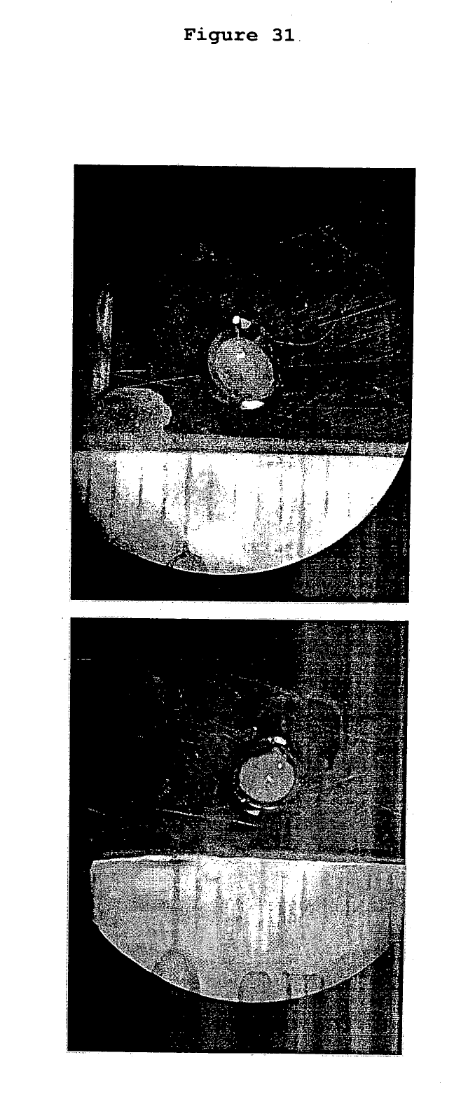

FIG. 32 Chitosan Beads. 1:3 ratio of PRP to 2.5% Chitosan solution (5× magnification) (Φ 2.5-3 mm).

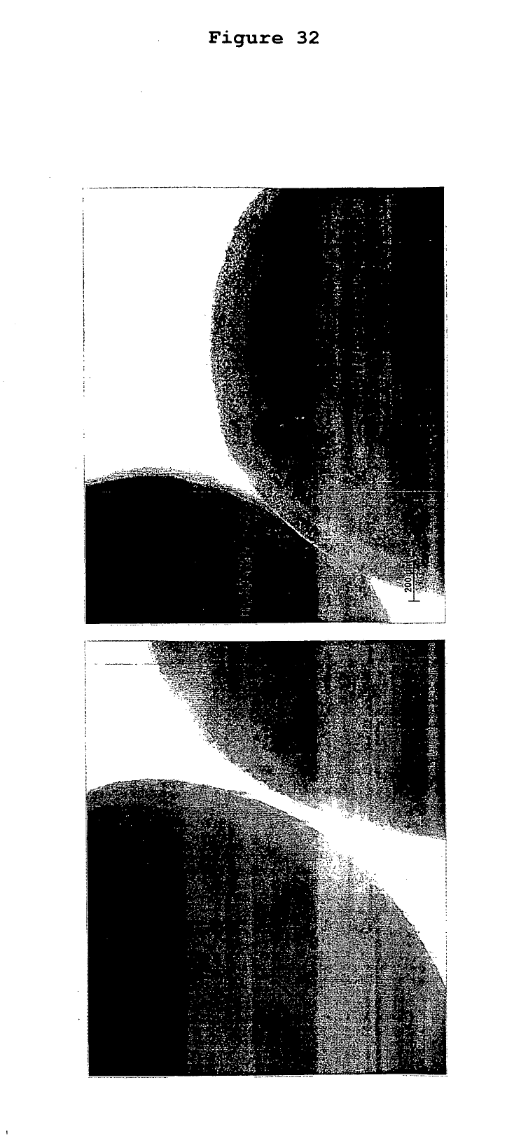

FIG. 33 Formation of Chitosan Capsules.

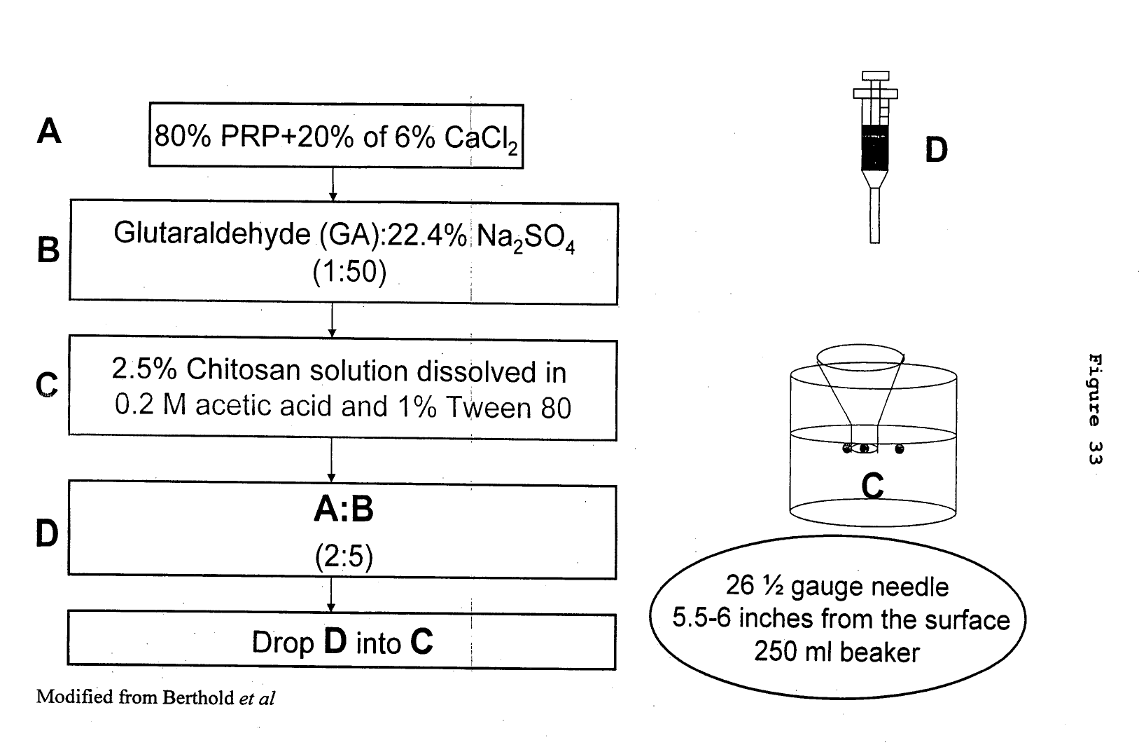

FIG. 34 Chitosan Capsules. 1:3 ratio of PRP to 22.4% NA₂SO₄ solution (φ 4.5-5 mm).

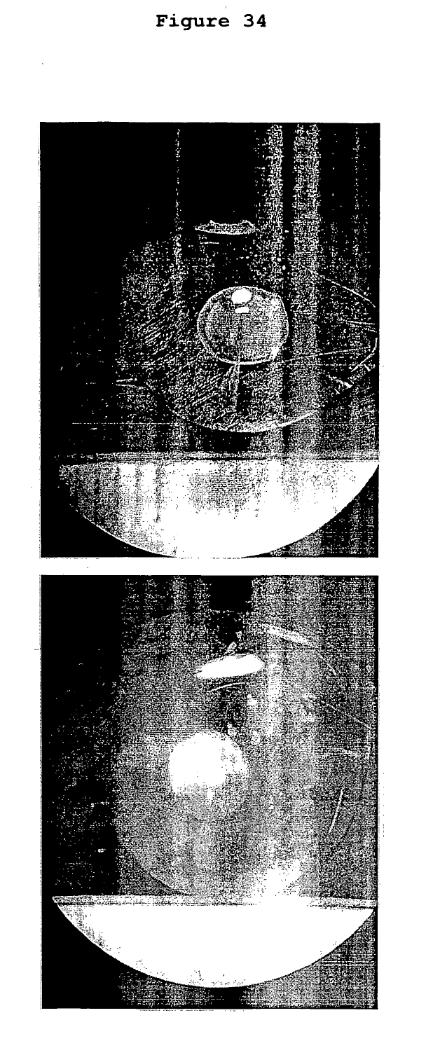

### DETAILED DESCRIPTION OF THE INVENTION

Definitions

“Autologous” shall mean, with respect to any of the instant methods, originating from the subject on whom the instant method is being practiced.

“Bone regeneration-facilitating material” shall mean a solid material which, when placed in, or in juxtaposition to, living bone under suitable conditions, serves as a scaffold for the formation of new bone by bone-forming cells. Bone-forming material includes, without limitation, collagen, bioglass (e.g., 45S5 BioGlass), BioOss (calcium phosphate-based bone graft substitute), Pepgen P-15 (synthetic P-15 peptide bound to a natural form of hydroxylapatite) and AlloGraft (demineralized bone matrix, allograft-based bone graft substitute).

“Bone formation-requiring site” shall mean a site on or in the bone of a subject where the formation of bone is desired. A bone formation-requiring site includes, for example, a space or recess formed in bone through decay or surgical bone removal. Such site can exist on or in any bone (e.g., maxillofacial or vertebral) in any subject.

“Calcium”, with regard to its use in the instant invention, shall mean calcium ions, which exist together with one or more types of negative ions. In one embodiment, calcium exists in the form of a CaCl₂ solution.

“Added growth factor” shall mean a growth factor which does not originate from the platelet-rich plasma used in the instant invention. For example, human PDGF added to human platelet-rich plasma constitutes exogenous growth factor, as opposed to the PDGF already in (i.e., originating from and hence endogenous to) the platelet-rich plasma.

“Added thrombin” shall mean thrombin which does not originate from the platelet-rich plasma used in the instant invention.

“Facilitating”, with respect to bone formation, is synonymous with “enhancing”, and shall mean permitting and/or increasing the rate of bone formation.

“PAR” shall mean thrombin-binding, G protein-coupled protease-activated receptor whose amino terminus is cleaved by thrombin.

“PAR-activating agent” shall mean an agent which binds to PAR, resulting in its activation in the form of a transmembrane signal.

“Platelet-originating growth factor” shall mean a growth factor which is naturally produced by and secreted from platelets. Examples of platelet-originating growth factors include platelet-derived growth factor and transforming growth factor beta.

“Platelet-rich plasma,” also referred to in the art as “PRP,” shall mean plasma having therein platelets at a concentration which exceeds the concentration of platelets usually found in whole plasma (i.e., plasma whose components have not been altered, diminished or removed). In one embodiment, platelet-rich plasma has a platelet concentration of between about 300% and 700% greater than the concentration of platelets in whole plasma. In another embodiment, platelet-rich plasma further comprises agents not naturally found in plasma, such as TRAP-6. In yet another embodiment, platelet-rich plasma further comprises TRAP-6 but is free from exogenous thrombin.

“Protein-permeable material” shall mean material that permits permeation by a protein of, or less than, a predetermined molecular weight, which permeation occurs at a rate slower than that at which water permeates the material. In one embodiment, the protein-permeable material is a calcium alginate gel or a chitosan gel which permits the permeation of platelet-derived growth factor.

“Subject” shall mean any organism including, without limitation, a mammal such as a mouse, a rat, a dog, a guinea pig, a ferret, a rabbit and a primate. In the preferred embodiment, the subject is a human being.

“Trap-6”, also referred to as “TRAP-6” and “TRAP”, shall mean thrombin receptor activator peptide-6 having the amino acid sequence SFLLRN.

### EMBODIMENTS OF THE INVENTION

This invention provides an article of manufacture comprising a capsule of protein-permeable material having platelet-rich plasma therein. In one embodiment, the platelet-rich plasma is human platelet-rich plasma. In another embodiment, the protein-permeable material is calcium alginate gel. In another embodiment, the protein-permeable material is chitosan gel. In another embodiment, the platelet-rich plasma further comprises a PAR-activating agent. In another embodiment, the PAR-activating agent is TRAP-6. In another embodiment, the article has a diameter of between about 2 mm and about 5 mm, and the protein-permeable material has a thickness of between about 0.4 mm and 0.8 mm. In another embodiment, the platelet-rich plasma further comprises an added growth factor. In another embodiment, the added growth factor is selected from the group consisting of platelet-derived growth factor, bone morphogenetic protein, transforming growth factor beta, insulin-like growth factor, epidermal growth factor, epithelial cell growth factor and vascular endothelial growth factor. In another embodiment, the added growth factor is platelet-derived growth factor or transforming growth factor beta. In another embodiment, the platelet-rich plasma further comprises a bone regeneration-facilitating material. In another embodiment, the bone regeneration-facilitating material is selected from the group consisting of collagen, BioOss, PepGen P-15, AlloGro, 45S5 BioGlass and autologous bone.

This invention also provides an article of manufacture comprising a porous bead having releasably contained therein (i) platelet-rich plasma and/or (ii) a growth factor. In one embodiment, the platelet-rich plasma is human platelet-rich plasma. In another embodiment, the porous bead comprises calcium alginate gel. In another embodiment, the porous bead comprises chitosan gel. In another embodiment, the platelet-rich plasma further comprises a PAR-activating agent. In another embodiment, the PAR-activating agent is TRAP-6. In another embodiment, the bead has a diameter of between about 2 mm and about 5 mm. In another embodiment, the growth factor is selected from the group consisting of platelet-derived growth factor, bone morphogenetic protein, transforming growth factor beta, insulin-like growth factor, epidermal growth factor, epithelial cell growth factor and vascular endothelial growth factor. In another embodiment, the growth factor is platelet-derived growth factor or transforming growth factor beta.

This invention further provides a composition of matter comprising (a) a capsule of protein-permeable material having a growth factor therein, (b) a porous bead having a growth factor releasably contained therein, and (c) a gel comprising platelet-rich plasma and a bone regeneration-facilitating material. In one embodiment, the platelet-rich plasma is human platelet-rich plasma. In another embodiment, the composition further comprises a PAR-activating agent. In another embodiment, the PAR-activating agent is TRAP-6. In another embodiment, the bead and capsule each has a diameter of between about 2 mm and about 5 mm. In another embodiment, the growth factors in the capsule and bead are different, and are selected from the group consisting of platelet-derived growth factor, bone morphogenetic protein, transforming growth factor beta, insulin-like growth factor, epidermal growth factor, epithelial cell growth factor and vascular endothelial growth factor. In another embodiment, the growth factors are platelet-derived growth factor and transforming growth factor beta. In another embodiment, the bone regeneration-facilitating material is selected from the group consisting of collagen, BioOss, PepGen P-15, AlloGro, 45S5 BioGlass and autologous bone. In another embodiment, the bone regeneration-facilitating material is collagen.

This invention further provides a method for making an article of manufacture comprising a capsule of protein-permeable material having platelet-rich plasma therein, which method comprises admixing platelet-rich plasma dropwise, under suitable conditions, with a material which, when solidified under such conditions, forms a protein-permeable capsule. In one embodiment, the platelet-rich plasma is human platelet-rich plasma. In another embodiment, (a) the material which, when solidified, forms a protein-permeable material comprises alginate, and (b) the platelet-rich plasma further comprises calcium, whereby calcium alginate gel is formed upon contact between the material and the platelet-rich plasma. In another embodiment, the platelet-rich plasma further comprises a PAR-activating agent. In another embodiment, the PAR-activating agent is TRAP-6. In another embodiment, the article has a diameter of between about 2 mm and about 5 mm, and the protein-permeable material has a thickness of between about 0.4 mm and 0.8 mm. In another embodiment, the platelet-rich plasma further comprises an added growth factor. In another embodiment, the added growth factor is selected from the group consisting of platelet-derived growth factor, bone morphogenetic protein, transforming growth factor beta, insulin-like growth factor, epidermal growth factor, epithelial cell growth factor, and vascular endothelial growth factor. In another embodiment, the added growth factor is platelet-derived growth factor or transforming growth factor beta. In another embodiment, the platelet-rich plasma further comprises a bone regeneration-facilitating material. In another embodiment, the bone regeneration-facilitating material is selected from the group consisting of collagen, BioOss, PepGen P-15, AlloGro, 45S5 BioGlass and autologous bone.

This invention further provides a method for facilitating bone formation in a subject comprising delivering to a bone formation-requiring site in the subject an article of manufacture comprising a capsule of protein-permeable material having platelet-rich plasma therein, wherein the platelet-rich plasma in the article is autologous. In one embodiment, the subject is human.

This invention further provides a method for facilitating bone formation in a subject comprising delivering to a bone formation-requiring site in the subject an article of manufacture of comprising a porous bead having autologous platelet-rich plasma releasably contained therein. In one embodiment, the subject is human.

This invention further provides a method for facilitating bone formation in a subject comprising delivering to a bone formation-requiring site in the subject a composition of matter comprising (a) a capsule of protein-permeable material having a growth factor therein, (b) a porous bead having a growth factor releasably contained therein, and (c) a gel comprising platelet-rich plasma and a bone regeneration-facilitating material, wherein the platelet-rich plasma in the composition is autologous. In one embodiment, the subject is human.

This invention further provides a method for delivering a platelet-originating growth factor to a subject at a location in the subject where delivery of the growth factor is desired comprising delivering to the site in the subject a capsule of protein-permeable material having autologous platelet-rich plasma therein, so as to permit the platelet-originating growth factor to be released from the platelets in the platelet-rich plasma and then be released from the capsule, thereby delivering the platelet-originating growth factor to the subject at the location where delivery of the growth factor is desired. In one embodiment, the subject is human.

This invention further provides a method for delivering a platelet-originating growth factor to a subject at a location in the subject where delivery of the growth factor is desired comprising delivering to the site in the subject a porous bead having autologous platelet-rich plasma releasably contained therein, so as to permit the platelet-originating growth factor to be released from the platelets in the platelet-rich plasma and then be released from the bead, thereby delivering the platelet-originating growth factor to the subject at the location where delivery of the growth factor is desired. In one embodiment, the subject is human.

Finally, this invention provides an article of manufacture comprising a packaging material having therein, in separate compartments, calcium and a material which, when solidified under suitable conditions, forms a protein-permeable capsule. In one embodiment, the material comprises alginate. In another embodiment, the material comprises chitosan. In another embodiment, the article further comprises (a) a PAR-activating agent, (b) a bone regeneration-facilitating material, (c) one or more growth factors, and/or (d) container(s), reagent(s) and an apparatus for preparing platelet-rich plasma and, using the platelet-rich plasma so prepared, admixing the platelet-rich plasma with the material which, when solidified under suitable conditions, forms a protein-permeable capsule, so as to form an article of manufacture comprising a capsule of protein-permeable material having platelet-rich plasma therein. In another embodiment, the PAR-activating agent is TRAP-6. In another embodiment, the bone regeneration-facilitating material is selected from the group consisting of collagen, BioOss, PepGen P-15, AlloGro, 45S5 BioGlass and autologous bone. In another embodiment, the growth factor is selected from the group consisting of platelet-derived growth factor, bone morphogenetic protein, transforming growth factor beta, insulin-like growth factor, epidermal growth factor, epithelial cell growth factor and vascular endothelial growth factor. In another embodiment, the growth factor is platelet-derived growth factor or transforming growth factor beta. In another embodiment, the article further comprises one or more porous beads capable of releasably containing therein (i) platelet-rich plasma and/or (ii) a growth factor. In another embodiment, the article further comprises instructions for use in facilitating bone formation in a subject.

This invention will be better understood from the Experimental Details which follow. However, one skilled in the art will readily appreciate that the specific methods and results discussed are merely illustrative of the invention as described more fully in the claims which follow thereafter.

### EXPERIMENTAL DETAILS

### Example 1

Synopsis

Platelet-rich plasma (PRP) is derived from an autogenous (i.e., autologous) preparation of concentrated platelets and contains growth factors such as platelet-derived growth factor (PDGF) and transforming growth factor beta (TGF-β). Clinical efficacy of PRP is dependant on increasing bioavailability of these factors and modulating release sequence and kinetics to match the rate of bone regeneration. The objective was to make a hydrogel-based, PRP-encapsulation system, which will delay growth factor release and extend their bioavailability. PRP was prepared by modifying the methods of Landesberg et al. The PRP was then re-suspended in 6% CaCl₂ and dispensed drop-wise into 1% alginate to form capsules. PDGF-AB release from these capsules was evaluated after 1, 3, 7 days (n=3), and growth factor levels were quantified by ELISA. It was found that capsule morphology and membrane integrity were maintained over time with uniform size (4.22±0.37 mm by 3.09±0.21 mm) and wall thickness 0.61±0.05 mm. PDGF-AB in supernatant increased significantly as incubation time increased (p<0.05) while the rate of release from the capsules was significantly lower as compared to thrombin controls. Results suggest that controlled release of PRP-derived growth factors can be achieved by PRP-encapsulation within a semi-permeable hydrogel membrane, permitting prolonged and modulated release.

Introduction

Knowledge of growth factor levels in PRP samples is necessary to ensure reliable and reproducible use of PRP for clinical treatment, since the regenerative potency of PRP depends on its growth factor levels (1). The potential of PRP to increase the rate of bone regeneration is also affected by the release rate of necessary growth factors. Quantification of growth factor levels shows that they diminish over a period of time; the life span of a platelet in a wound and the period of the direct influence of its growth factors are less than 5 days (4). Controlling the release rate will prolong the lifetime of PRP as a useful bone regeneration tool.

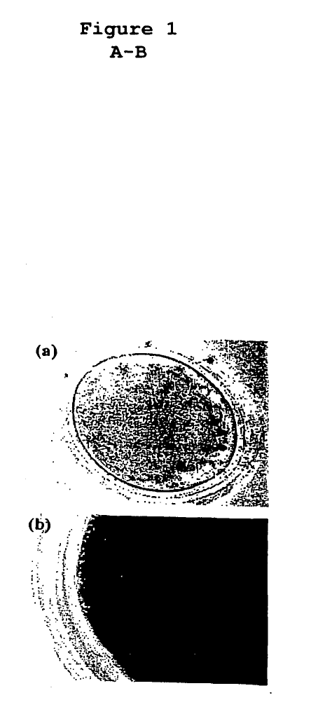

A significant challenge lies in increasing bioavailability of these growth factors, modulating release sequence and kinetics to match the rate of bone regeneration. The approach here is to engineer a PRP encapsulation system able to support prolonged and controlled release of relevant growth factors. Effective immobilization of PRP can be achieved by enclosing a large number of particles, in an aqueous solution, inside a semi-permeable membrane capsule to control the rate of growth factor release. Encapsulation in calcium alginate gels is advantageous due to the various properties of alginate, including: (1) relatively inert aqueous environment within matrix; (2) mild room temperature encapsulation process free of organic solvents; (3) high gel porosity, allowing for high diffusion rates of macromolecules; (4) ability to control porosity with simple coating procedures; and (5) dissolution and biodegradation under normal physiological conditions (10). The basic method behind alginate capsule synthesis is the gelation of the alginate solution with bivalent cations. Once the cationic solution containing PRP is dropped into the anionic alginate solution, a capsular membrane forms instantaneously around the droplet via polymer cross-linking. Changing the gelation conditions makes it possible to easily control some of the capsule characteristics, such as diameter or wall thickness, rate of degradation, and permeability. The parameters in Table 1 were varied during encapsulation procedures and produced capsules of spherical and elliptical morphology shown in FIG. 1 with uniform size and wall thickness.

TABLE 1
Encapsulation parameters during preliminary experiments

| Stirring speed | Ranged from |
|---|---|
| | 200-1000 rpm |
| Dropping height | Ranged from 5-12 |
| between needle tip and | inches |
| surface of alginate | |
| Concentration of | 0.5%, 1% |
| alginate | |
| Concentration of CaCl₂ | 4%, 6% |
| Concentration of | 5%, 10% |
| dextran | |
| Ratio of PRP to CaCl₂ | 1:3, 2:5, 1:1 |

The main objective of this study was to evaluate PRP encapsulation and growth factor release kinetics in an alginate gel-based system. It is now believed that PRP encapsulation delays the rate of growth factor release and extends their bioavailability.

Materials and Methods

PRP was prepared by a modification of Landesberg et al (4). Sixty milliliters of venous blood from healthy adult volunteers were mixed with ACD Solution B in 9.0 ml vacutainer tubes (Becton Dickinson, Franklin Lakes, N.J.). The ACD solution contained 13.2 g/L trisodium citrate; 4.8 g/L citric acid, and 14.7 g/L dextrose. The samples were centrifuged at 2000 rpm for 10 minutes (ACE Surgical Supply Company, Inc; Brockton, Mass.). The plasma and buffy coat layers were removed and placed into 5 ml tubes, and tubes were spun at 2000 rpm for an additional 15 minutes. The upper half of the preparation was designated platelet-poor plasma (PPP) and subsequently discarded. The lower half of the plasma and the pellet were re-suspended and pooled to be the platelet-rich plasma (PRP).

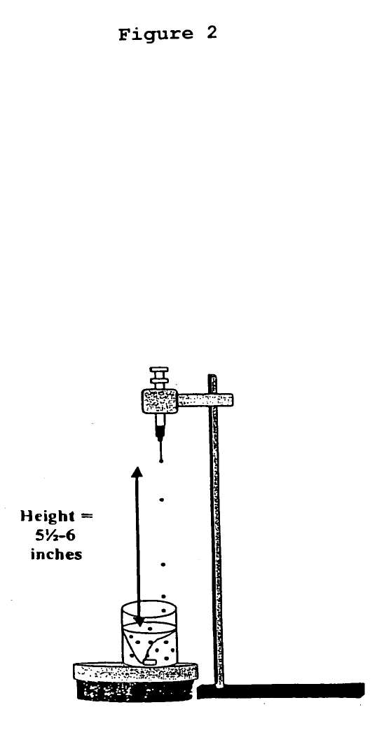

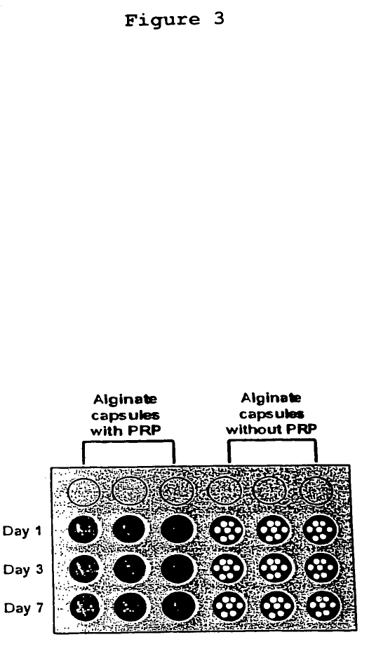

The PRP was re-suspended in 6% CaCl₂ solution in a 2:5 ratio, and dispensed drop-wise via a 26%-gauge syringe needle into a 1% alginate solution (Sigma, St. Louis, Mo.). The alginate solution was maintained under constant stirring at low speed (600-900 rpm), using a magnetic stirrer with the vortex situated near the wall of the beaker in order to keep the droplets from sticking together. Constant stirring was maintained for approximately 1 minute once the capsules were formed. A schematic of the apparatus used to encapsulate the PRP is shown below in FIG. 2. The morphology and dimensions of the alginate-PRP capsules were determined post-fabrication. To evaluate growth factor release, the alginate-PRP capsules were incubated in Dulbecco's Modification of Eagle's Medium (DMEM, Mediatech, Herdon, Va.) without serum for 1, 3, 7 days (n=3), as shown in FIG. 3. Unencapsulated PRP with thrombin and alginate capsules without PRP served as controls. All collected supernatant samples were stored at −70° C. prior to analyses.

Supernatant and capsules collected from all time points were assayed for PDGF-AB content using a diagnostic kit from R&D Systems (Minneapolis, Minn.), an assay based on a sandwich enzyme immunoassay technique. Prior to analyses, alginate was dissolved in sodium citrate at 4° C. The PDGF-AB assay used a pre-coated microtiter plate with a monoclonal antibody to PDGF-AA. Preparation and dilution of samples and standards were performed as directed by the manufacturer. Both the standards and the samples were incubated for 3 hours at room temperature. The plate was washed with buffer and a conjugated antibody to PDGF-BB was added to the wells and incubated at room temperature for 1 additional hour. The plate was then washed and substrate was added for 20 minutes at room temperature. The reaction was stopped and absorbance was determined at 450 nm using a spectrophotometer (SpectraFluor Plus, Tecan, Maennedorf, Switzerland). A standard curve was generated and the PDGF-AB levels (pg/ml) of each sample were determined, and the total amount of growth factors were calculated based on the amount of supernatant obtained after clot retraction.

All results were expressed as mean±standard deviation. Multi-way analysis of variance (ANOVA) was performed and the Tukey-Kramer test was used to compare means. Significance was determined at p<0.05.

Results

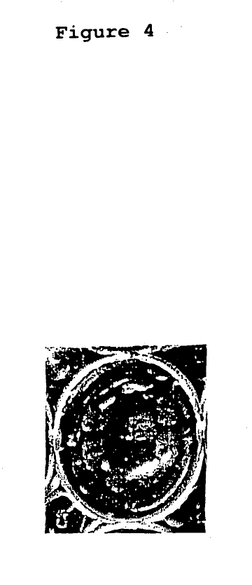

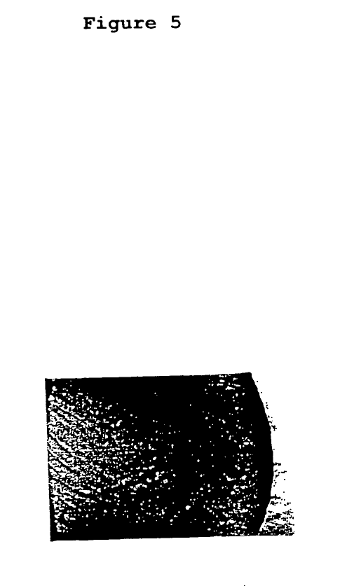

The alginate-PRP capsules were found to be uniform in size (4.22±0.37 mm by 3.09±0.21 mm), with a capsule wall thickness of 0.61±0.05 mm. Capsule morphology and membrane integrity were maintained over time with representative images shown in FIGS. 4 and 5.

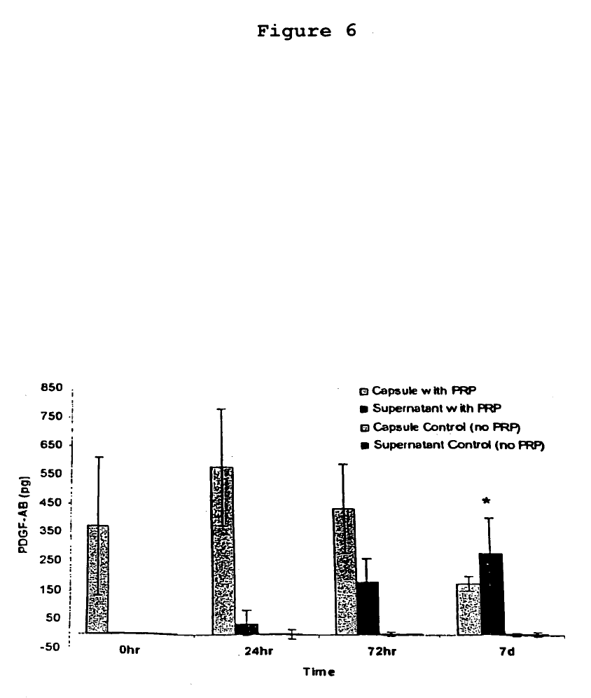

As shown in FIG. 6, the quantity of PDGF-AB within the capsules decreased as it was released into the media, while the amount of PDGF-AB in the supernatant increased significantly as incubation time increased (p<0.05). In the thrombin control, the majority of PDGF-AB was released within 24 hours; additionally, the rate of PDGF-AB release was significantly lower from the capsule as compared to controls.

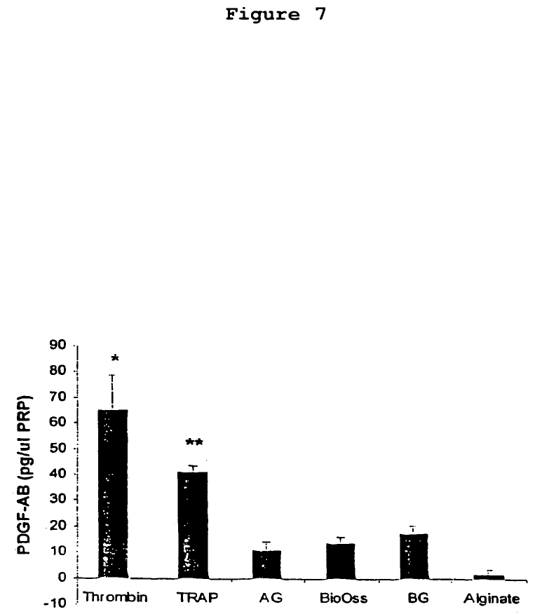

FIG. 7 compares the normalized effects of substrates (including alginate gel) on PDGF-AB released per microliter of PRP at 24 hours. It was observed that the thrombin and TRAP groups had the highest release of PDGF-AB per microliter of PRP while the alginate capsules had the lowest release. There were no significant differences among the AlloGro (AG), BioOss (BO), or BioGlass (BG) groups.

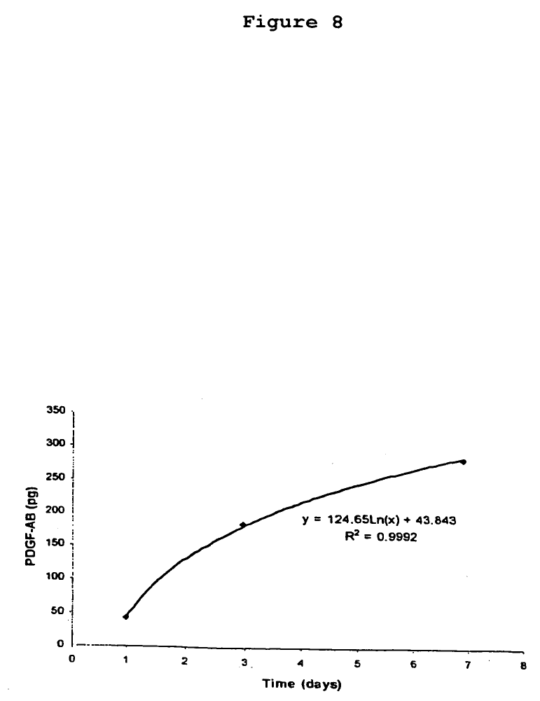

The release kinetics in FIG. 8 shows that the quantity of PDGF-AB in the supernatant increased logarithmically with time over 7 days. As incubation time increased, the amount of PDGF-AB released from the capsules increased, confirming that the alginate membrane retained this particular growth factor and prolonged its release.

Discussion

Several factors and limitations could affect the formation of spherical capsules. These include the dropping height of the PRP, stirring speed of the alginate solution, as well as the concentration of the cationic and anionic solutions. Changing the gelation conditions makes it possible to easily control some of the capsule characteristics, such as diameter or wall thickness, rate of degradation, permeability, and porosity. Slow stirring speeds or low dropping heights could form capsules with “tails” or non-uniform capsules. Although spherical capsules of uniform size were formed under non-sterile conditions in preliminary trials, the same results were not consistently reproduced for the incubation experiment, possible due to laminar flow of the hood under sterile conditions. In addition, after PRP is prepared or when PRP is combined with the cationic solution, there is only a certain amount of storage time available before PRP begins to degrade, thus losing its effectiveness to deliver growth factors or be encapsulated. It is debatable whether alginate will fully degrade once it is implanted for dental applications. It is suggested that biological degradation of alginate occurs via enzymes (alginate lyases) that also operate through the alkaline β-elimination mechanism (7). However, degradation rate may also depend on physiological pH as well as the molecular weight of the alginate. Porosity and wall thickness of the alginate capsule could hinder growth factor release rate as well, thus making the determination of optimal parameters extremely important.

Comparison of various clotting substrates (thrombin, TRAP, AlloGro, BioOss, and bioactive glass) to the alginate capsules at 24 hours showed that the lowest release of PDGF-AB was from the capsules, although this was not statistically significant. While TGFβ and IGF-1 were not tested in this study, they are known to be components of PRP and are critical to osteoblast differentiation in the later stages of bone regeneration, making it extremely important to delay their release. TGFβ and IGF-1 are smaller than PDGF (PDGF 30 kD; TGFβ24 kD; IGF-1 7.6 kD) (9) and may have a different release profile than the growth factor tested.

Researchers are currently exploring the area of PRP as well as various encapsulation methods for drug release applications (2, 4, 5, 8). Landesberg et al. compared two methods of preparing PRP gel and the levels of PDGF and TGF-β in each preparation. They found that both methods of preparation yielded PRP gel in less than 30 minutes and the levels of PDGF and TGF-β were similar regardless of which method was used for initiation of clot formation. They also noted that although a significant amount of clot retraction was achieved by 2 hours, the maximal retraction takes place by 24 hours. While there may be growth factors/cytokines remaining within the clot, enzymatic breakdown by fibrinolysis would be necessary for their release. Their method of assaying the two growth factors allowed the amounts available to the surrounding bone and tissue surfaces to be quantitated. Marx et al. developed a gradient density centrifugation technique that produced a concentration of human platelets of 338% and identified PDGF and TGF-β within them. Cancellous cellular marrow grafts demonstrated cells capable of responding to the growth factors by bearing cell membrane receptors. The additional amounts of these growth factors obtained by adding PRP to grafts demonstrated a maturation rate 1.62-2.16 times that of grafts with PRP. There was a greater bone density in grafts in which PRP was added than in grafts in which PRP was not added.

Blandino et al. studied the diffusion of an enzyme of high molecular weight out of calcium alginate gel capsules, obtained at various sodium alginate and CaCl₂ concentrations. The authors found that an increase in the concentration of sodium alginate and CaCl₂ gave rise to a reduction in the enzyme leakage over time. It was shown that the rate of enzyme release at a given time and CaCl₂ concentration depended on the sodium alginate concentration and thus on capsule membrane thickness and degree of cross-linking. On increasing the alginate concentration, the number of apparent cross-linking points increased, resulting in the decreased mesh size within the gel. Additionally, Kikuchi et al. investigated the release of macromolecular drug from calcium-alginate gel beads. Dextran release was observed to be molecular weight-dependent where FITC-dex release was retarded as the molecular weight of FITC-dex increased from 9,400 to 145,000. Release of a lower molecular weight dextran was mainly governed by the drug diffusion through the calcium-alginate gel matrix. It was found that the release of dextran with a molecular weight of 9,400 was proportional to the square root of time for up to the first 60% of release. With increasing dextran molecular weights, the release was strongly influenced by the dissolution of the gel matrix and the release pattern became sigmoidal. For FITC-dex with molecular weight of 145,000, the release coincided with alginate gel disintegration due to ion exchange. Minimal dextran release was observed at pH 1.2, while rapid dextran release within a narrow time range was achieved at pH 6.8. The instant results suggest that calcium-alginate is a useful vehicle for oral drug delivery.

Conclusion

Platelet-rich plasma was encapsulated in a calcium alginate gel to form a semi-spherical morphology using appropriate gelation parameters (1% alginate with 6% CaCl₂ at a dropping height of approximately 5½-6 inches). Controlled release of PRP-related growth factors can be achieved by PRP encapsulation within a semi-permeable membrane. This novel hydrogel delivery system permits prolonged and modulated release of growth factors relevant for bone regeneration.

### REFERENCES FOR EXAMPLE 1 AND BACKGROUND OF THE INVENTION

1. Weibrich, G., et al., “Growth factor levels in platelet-rich plasma and correlations with donor age, sex, and platelet count”. Journal of Cranio-Maxillofacial Surgery, 30:97-102 (2002).
2. Landesberg, R., et al., “Quantification of Growth Factor Levels Using a Simplified Method of Platelet-Rich Plasma Gel Preparation”. J. Oral Maxillofac. Surg, 58:297-300 (2000).
3. Carlson, N. E. and Roach, R. B., “Platelet-rich plasma Clinical applications in dentistry”. JADA, 133:1383-1386 (2002).
4. Marx, R. E., et al., “Platelet-rich plasma Growth factor enhancement for bone grafts”. Oral Surg. Oral Med. Oral Pathol. Oral Radiol. Endod. 85:638-646 (1998).
5. Blandino, A., et al., “Glucose oxidase release from calcium alginate gel capsules”. Enzyme and Microbial Technology, 27:319-324 (2000).
6. Beguin, S. and Keularts, I. “On the Coagulation of Platelet-Rich Plasma Physiological Mechanism and Pharmacological Consequences”. Haemostasis, 29:50-57 (1999).
7. Christensen, B. E., et al., “Stability and Degradation of Alginates”. (1995)
8. Kikuchi, A., et al., “Effect of Ca²⁺-alginate gel dissolution on release of dextran with different molecular weights”. Journal of Controlled Release, 58:21-28 (1999).
9. Papadopoulos, S., et al., “Protein Diffusion in Living Skeletal Muscle Fibers: Dependence on Protein Size, Fiber Type, and Contraction”. Biophysical Journal, 79:2084-2094 (2000).
10. Gombotz, W. R. and Wee, S. F., “Protein release from alginate matrices”. Advanced Drug Delivery Reviews, 31:267-285 (1998).

### Example 2

Chitosan and its Application in Tissue Engineering

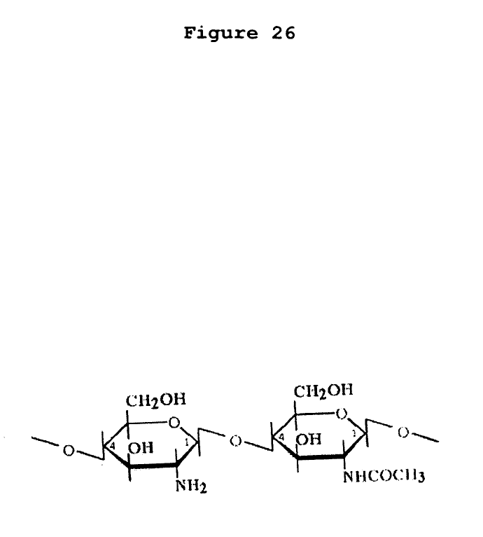

Chitosan is a polysaccharide biopolymer derived from chitin. Chitin is found primarily in the exoskeleton of arthropods such as crustaceans, and next to cellulose, chitin is the second most abundant polymer found in nature [1]. Chitosan is formed by deacetylating chitin. Both chitin and chitosan molecule consists of a co-polymer of N-acetyl-glucosamine and N-glucosamine (FIG. 26), and the monomers are arranged randomly or distributed in blocks throughout polymer. When the number of N-glucosamine monomers exceeds 50%, the biopolymer is termed chitosan [2], and when it is below 50%, the polymer is classified as chitin.

Chitosan can be produced with a wide range of molecular weights and average degrees of deacetylation. It has been shown to augment the immune response against bacteria, viruses and cancerous cells [3;4]. It has been used as fruit coating to prevent bacterial growth [5]. Although the precise mechanism behind the antibacterial potential of chitosan is not fully understood, it is proposed that an inhibition of bacterial mRNA synthesis is achieved via the interaction of chitosan with DNA. Although the chitosan molecule itself is too large to pass through a cell membrane, it may be hydrolyzed by host hydrolytic enzymes such as chitinase. These smaller molecules of hydrolyzed chitosan could penetrate to the nuclei of the fungus where they could interfere with mRNA synthesis. Chitosan can also be degraded by enzymatic hydrolysis through the actions of lysozomes [6]. The degradation rate increases with decreasing degree of deacetylation. While the antibacterial potential of chitosan is not a focus of this application, it was an important criterion for material selection and its relevance in preventing pulp re-infection will be investigated in future studies.

Chitosan is a widely used research natural biomaterial which has also been considered for biomedical applications, e.g. wound healing [7-9], bone [10-12] and cartilage tissue engineering [13-15]. The wound healing potential of chitosan is believed to be derived largely from its sugar N-acetylglucosamine [16]. Bulk and surface modifications of chitosan have been performed in order to make the material more favorable for bone regeneration [11;17]. When Malette et al. used chitosan for bone healing in a radii canine model, they found that chitosan improved bone formation by promoting the regeneration of marrow through the cortex [18]. Chitosan has also been considered for dental application in both animal and human studies. Muzzarelli et al. [19] treated 52 cases of periodontitis with chitosan gels, and found a significant reduction in tooth mobility and pocket depth as well as an enhancement in the regeneration of architectural organization.

Recently chitosan has been investigated for oral wound healing in humans. Sapelli et al. applied chitosan powder in periodontal pockets, palatal wounds and extraction sockets. Significant wound healing was observed with the matrix group [20]. These reports suggest that chitosan is a promising material for dental tissue engineering.

### REFERENCES FOR EXAMPLE 2

1. Choi S H, Kim C K, Cho K S, Huh J S, Sorensen R G, Wozney J M, Wikesjo U M (2002) Effect of recombinant human bone morphogenetic protein-2/absorbable collagen sponge (rhBMP-2/ACS) on healing in 3-wall intrabony defects in dogs. J. Periodontol. 73:63-72
2. Khor E, Lim L Y (2003) Implantable applications of chitin and chitosan. Biomaterials 24:2339-2349
3. Nishimura K, Nishimura S, Seo H, Nishi N, Tokura S, Azuma I (1987) Effect of multiporous microspheres derived from chitin and partially deacetylated chitin on the activation of mouse peritoneal macrophages. Vaccine 5:136-140
4. Klokkevold P R, Fukayama H, Sung E C, Bertolami C N (1999) The effect of chitosan (poly-N-acetyl glucosamine) on lingual hemostasis in heparinized rabbits. J. Oral Maxillofac. Surg. 57:49-52
5. Rabea E I, Badawy M E, Stevens C V, Smagghe G, Steurbaut W (2003) Chitosan as antimicrobial agent: applications and mode of action. Biomacromolecules. 4:1457-1465
6. Etienne O, Schneider A, Taddei C, Richert L, Schaaf P, Voegel J C, Egles C, Picart C (2005) Degradability of polysaccharides multilayer films in the oral environment: an in vitro and in vivo study. Biomacromolecules. 6.(2):726.-33., -Apr
7. Ma L, Gao C, Mao Z, Zhou J, Shen J, Hu X, Han C (2003) Collagen/chitosan porous scaffolds with improved biostability for skin tissue engineering. Biomaterials 24:4833-4841
8. Sarasam A, Madihally S V (2005) Characterization of chitosan-polycaprolactone blends for tissue engineering applications. Biomaterials 26:5500-5508
9. Mori T, Okumura M, Matsuura M, Ueno K, Tokura S, Okamoto Y, Minami S, Fujinaga T (1997) Effects of chitin and its derivatives on the proliferation and cytokine production of fibroblasts in vitro. Biomaterials 18:947-951
10. Park J S, Choi S H, Moon I S, Cho K S, Chai J K, Kim C K (2003) Eight-week histological analysis on the effect of chitosan on surgically created one-wall intrabony defects in beagle dogs. J. Clin. Periodontol. 30:443-453
11. Muzzarelli R A, Biagini G, Bellardini M, Simonelli L, Castaldini C, Fratto G (1993) Osteoconduction exerted by methylpyrrolidinone chitosan used in dental surgery. Biomaterials 14:39-43
12. Cho B C, Kim J Y, Lee J H, Chung H Y, Park J W, Roh K H, Kim G U, Kwon I C, Jang K H, Lee D S, Park N W, Kim I S (2004) The bone regenerative effect of chitosan microsphere-encapsulated growth hormone on bony consolidation in mandibular distraction osteogenesis in a dog model. Journal of Craniofacial. Surgery. 15.(2):299.-311.; discussion. 312.-3
13. Zielinski B A, Aebischer P (1994) Chitosan as a matrix for mammalian cell encapsulation. Biomaterials 15:1049-1056
14. Park D J, Choi B H, Zhu S J, Huh J Y, Kim B Y, Lee S H (2005) Injectable bone using chitosan-alginate gel/mesenchymal stem cells/BMP-2 composites. Journal of Cranio.-Maxillo.-Facial. Surgery. 33.(1):50.-4
15. Suh J K, Matthew H W (2000) Application of chitosan-based polysaccharide biomaterials in cartilage tissue engineering: a review. Biomaterials 21:2589-2598
16. Reynolds B (1960) Wound healing III: artificial maturation of arrested regenerate with an acetylated amino sugar. The American Surgeon 26:113-117
17. Jung U, Suh J, Choi S, Cho K, Chai J, Kim C (2000) The bone regenerative effect of chitsan on the calvarial critical size defect in Sprague-Dawley rats. Journal of Korean Academy of Periodontology 30:851-868
18. Malette W, Quigley H, Adickes E (1986) Chitin in nature and technology. In: Muzzarelli R, Jeuniaux C, Gooday G (eds) Chitosan effect in nature and technology. Plenum Press, New York, pp 435-442
19. Muzzarelli R, Biagini G, Pugnaloni A, Filippini O, Baldassarre V, Castaldini C, Rizzoli C (1989) Reconstruction of parodontal tissue with chitosan. Biomaterials 10:598-603
20. Sapelli P, Baldassare V, Muzzarelli R, Emanuelli M (1986) Chitosan in Dentistry. Chitin in Nature and Technology. pp 507-512

### Example 3

PRP Encapsulation and its Effect on Osteosarcoma Cells

The following experiments examine cell growth and activity in connection with PRP and alginate beads. It is believed that PRP-derived growth factors will have a positive effect on the growth and phenotypic expression of human osteoblast-like cells.

Materials and Methods

Control group: Human osteoblast-like cells (SaOS-2 human osteosarcoma line).

Experimental group: Human osteoblast-like cells (SaOS-2 human osteosarcoma line) with PRP+alginate beads.

Results

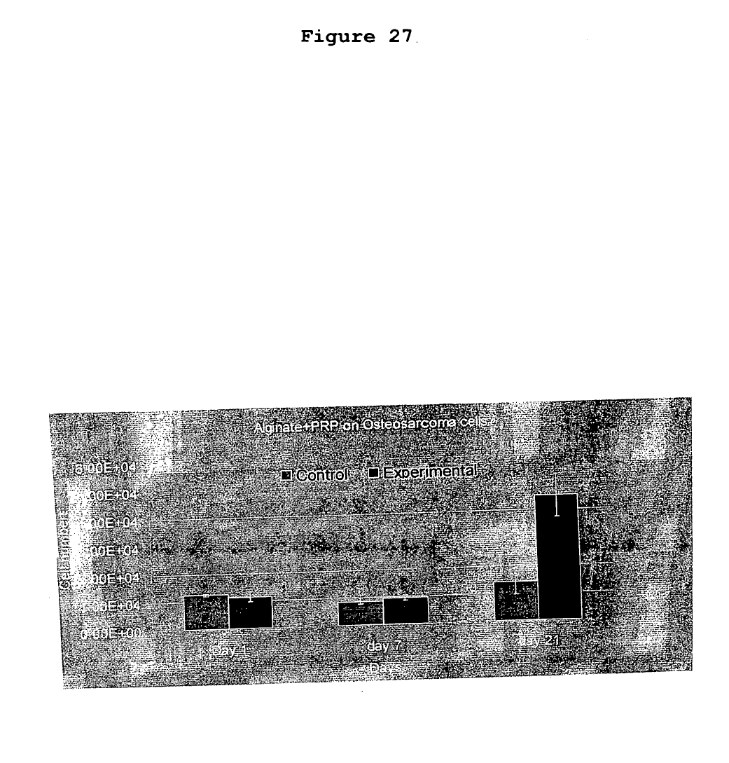

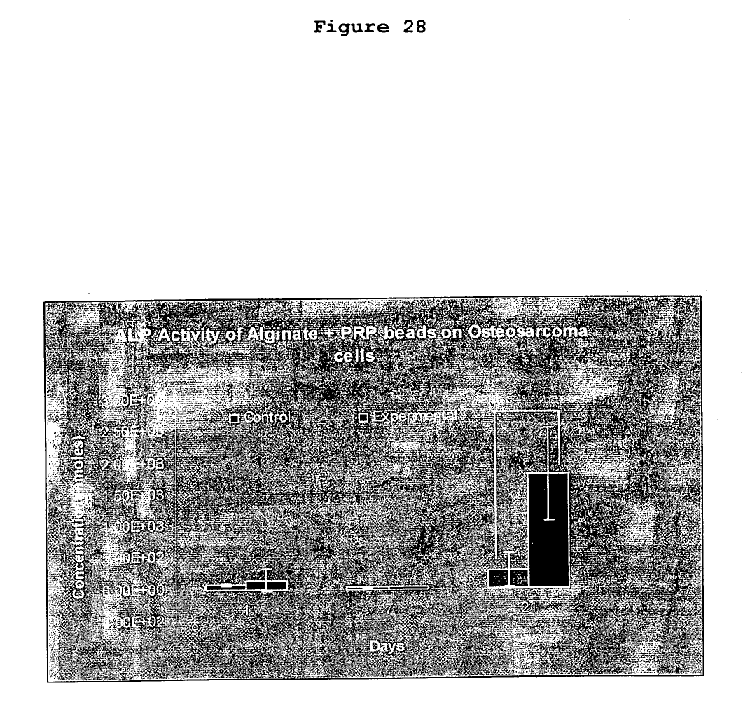

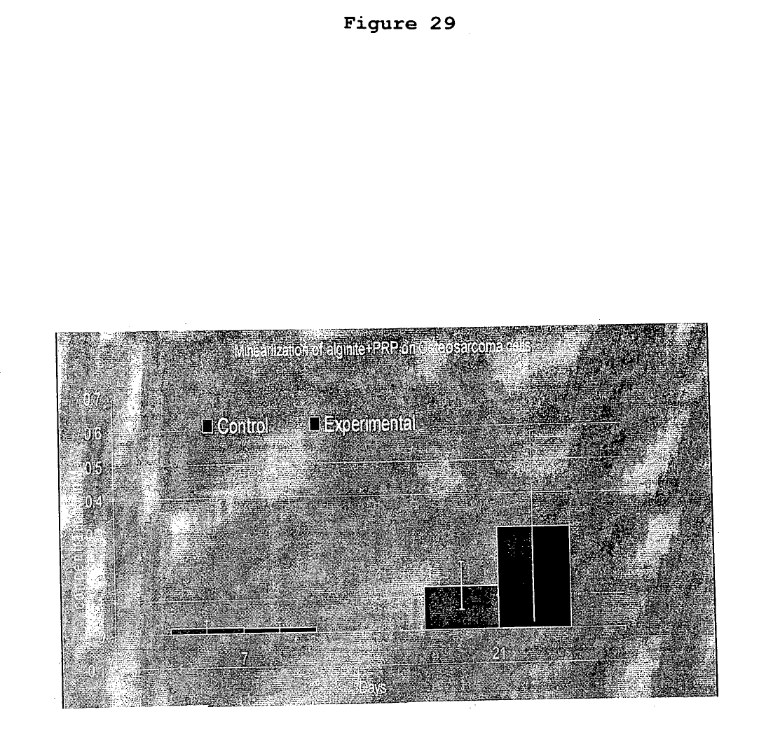

End Point Analysis: cell number (FIG. 27), ALP quantitative assay (n=6) (FIG. 28), mineralization quantitative (n=3) (FIG. 29) and mineralization qualitative (n=3).

1. An article of manufacture comprising a capsule of protein-permeable material having platelet-rich plasma therein.

2. The article of claim 1, wherein the platelet-rich plasma is human platelet-rich plasma.

3. The article of claim 1, wherein the protein-permeable material is calcium alginate gel.

4. The article of claim 1, wherein the protein-permeable material is chitosan gel.

5. The article of claim 1, wherein the platelet-rich plasma further comprises a PAR-activating agent.

6. The article of claim 5, wherein the PAR-activating agent is TRAP-6.

7. The article of claim 1, wherein the article has a diameter of between about 2 mm and about 5 mm, and the protein-permeable material has a thickness of between about 0.4 mm and 0.8 mm.

8. The article of claim 1, wherein the platelet-rich plasma further comprises an added growth factor.

9. The article of claim 8, wherein the added growth factor is selected from the group consisting of platelet-derived growth factor, bone morphogenetic protein, transforming growth factor beta, insulin-like growth factor, epidermal growth factor, epithelial cell growth factor and vascular endothelial growth factor.

10. The article of claim 9, wherein the added growth factor is platelet-derived growth factor or transforming growth factor beta.

11. The article of claim 1, wherein the platelet-rich plasma further comprises a bone regeneration-facilitating material.

12. The article of claim 11, wherein the bone regeneration-facilitating material is selected from the group consisting of collagen, BioOss, PepGen P-15, AlloGro, 45S5 BioGlass and autologous bone.

13. An article of manufacture comprising a porous bead having releasably contained therein (i) platelet-rich plasma and/or (ii) a growth factor.

14-21. (canceled)

22. A composition of matter comprising (a) a capsule of protein-permeable material having a growth factor therein, (b) a porous bead having a growth factor releasably contained therein, and (c) a gel comprising platelet-rich plasma and a bone regeneration-facilitating material.

23-61. (canceled)
